(19) United States
(12) Patent Application Publication
(10) Pub. No.: US 2003/0235872 A1
(43) Pub. Date: Dec. 25, 2003

(54) Assay method

(76) Inventors: David Henry Small, Ashburton (AU); Supundi Subasinghe, Endeavour Hills (AU); Marie-Isabel Aguilar, Warrandyte (AU)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/465,444

(22) Filed: Jun. 19, 2003

Related U.S. Application Data
(60) Provisional application No. 60/392,761, filed on Jul. 1, 2002.

(30) Foreign Application Priority Data
Jun. 20, 2002 (AU) PCT AU PS3064

(51) Int. Cl. G01N 33/53; G01N 33/567; C12M 1/34

(52) U.S. Cl. 435/7.2; 435/287.2

(57) ABSTRACT

Methods and devices for rapid screening of drug candidates, especially candidate agents for treatment of Alzheimer's disease and stroke are disclosed. The invention provides treatment compounds and a biosensor method and device which is particularly applicable to screening libraries of compounds.

### CROSS REFERENCE TO RELATED APPLICATIONS

This Application claims priority of U.S. Provisional Serial No. 60/392,761 filed Jul. 1, 2002, incorporated herein by reference.

### FIELD OF THE INVENTION

This invention relates to a method and device for rapid screening of drug candidates, especially candidate agents for treatment of Alzheimer's disease and stroke. The invention provides a biosensor method and device which is particularly applicable to screening libraries of compounds.

### BACKGROUND OF THE INVENTION

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

Alzheimer's disease (AD) is a progressive neurodegenerative disorder characterized by the accumulation of deposits of amyloid β protein (Aβ) in the form of amyloid plaques and cerebral amyloid angiopathy (CAA) (Price et al., 1991). Aβ is toxic, and accumulation of Aβ in the neuropil contributes to degenerative changes such as tangle formation and gliosis (Small et al., 2001). The accumulation of CAA causes the loss of cerebrovascular smooth muscle cells (SMCs) and weakening of the small and mid-sized vessels in the cerebral cortex and leptomeninges, and consequently is a risk factor for intracerebral haemorrhage (stroke), cerebral infarction, and dementia (Vinters, 1987; Ghiso and Frangione, 2001). In certain familial conditions amyloid is deposited predominantly as CAA (Ghiso and Frangione, 2001).

The major component of the amyloid deposits is a 4 kDa polypeptide known as β-amyloid protein (Aβ) (Glenner et al., 1984; Masters et al., 1985), which is derived from a much larger β-amyloid protein precursor (APP) (Kang et al., 1987). The major form of Aβ which is produced in the brain contains 40 amino acid residues. However, minor forms containing 42 or 43 residues are also formed. Production of these minor forms is closely linked to the pathogenesis of AD (Scheuner et al., 1996).

One approach to AD therapy is to inhibit production of Aβ in the brain. Proteolytic cleavage of APP by BACE1 and γ-secretase generates the full-length Aβ, which is then released from cells (Nunan and Small, 2000). Therefore inhibitors of either BACE1 or γ-secretase may be of therapeutic value. Alternatively, a number of studies have shown that cholesterol can influence Aβ release (Simons et al., 1998; Hartmann, 2001; Fassbender et al., 2001; Frears et al., 1999; Friedhoff et al., 2001). Therefore inhibitors of cholesterol biosynthesis, such as statins, may also be of therapeutic value. One advantage of statins is that they have relatively low toxicities, and their mode of action is much better understood than many other compounds currently being investigated as therapeutic agents for AD. However, there is some disagreement in the art as to the value of lowering cholesterol levels, and some workers consider that cholesterol is actually beneficial (Ji et al, 2002).

The mechanism of Aβ toxicity is poorly understood (Small et al., 2001). Aβ can bind to lipids (Curtain et al., 2001; Valdez-Gonzalez et al., 2001), including gangliosides (Ariga et al., 2001), sphingolipids (Mahfoud et al., 2002) and cholesterol (Kremer et al., 2000; Avdulov et al., 1997; Eckbert et al., 2000). In particular, Aβ can bind to membrane lipids, and this interaction may be toxic for cells (Hertel et al., 1997). However, few studies have attempted to correlate the degree of lipid binding by Aβ with its toxicity. Ji et al. (2002) have suggested that the binding of Aβ to cholesterol might prevent Aβ toxicity by inhibiting its oligomerization.

It is also known that acetylcholinesterase (AChE) colocalises with Aβ in the amyloid deposits which are found in the brains of Alzheimer's disease patients, and that AChE accelerates amyloid formation, both from wild-type Aβ and from a mutant Aβ peptide which alone is able to produce few amyloid-like fibres. This action of AChE is not affected by an inhibitor of the enzyme active site, but is inhibited by propidium, which binds to the peripheral anionic binding site. In contrast, butyrylcholinesterase, which lacks this peripheral anionic binding site, did not affect amyloid formation (Inestrosa et al. 1996). AChE forms stable complexes with Aβ can bind to the enzyme acetylcholinesterase to form stable complexes, and this binding increases the neurotoxicity of amyloid fibrils (Alvarez et al., 1998). Modelling studies suggest that a major hydrophobic sequence, designated Site I, which is exposed on the surface of AChE, is involved in complex formation with Aβ, and a 35 amino acid hydrophobic peptide corresponding to this sequence is indeed able to promote amyloid formation and is incorporated into growing Aβ fibrils (De Ferrari et al., 2001). Site I is also able to interact with liposomes (Shin et al., 1996).

In addition to amyloid deposits of the Aβ type, there are a number of other proteins which form amyloid deposits, and of these several are known to be involved in the pathogenesis of clinical conditions. For example immunoglobulin light chains form the amyloid fibrils in primary amyloidosis, and reactive or secondary amyloidosis is caused by a number of different proteins. Familial amyloidosis is caused by deposits of mutant forms of transthyretin, apolipoprotein A-I, gelsolin fibrinogen A α, or lysozyme; of these, the most common form of homelial amyloidosis is that caused by mutant transthyretin. These amyloidoses are all systemic, and rarely if ever involve the central nervous system. The systemic amyloidoses have recently been reviewed (Falk et al., 1997).

Deposition of amyloid-like fibrils may also be important in other neurodegenerative diseases, such as Parkinson's disease and other conditions in which α-synuclein fibrils are deposited. These include Parkinson's disease itself, dementia with Lewy body formation, multiple system atrophy, Hallerboden-Spatz disease, and diffuse Lewy body disease.

The technique of surface plasmon resonance (SPR)has been very widely applied to the analysis of the interaction between ligands and ligand-binding compounds in a variety of situations, and biosensors based on this principle are commercially available, for example from BIAcore AB and from Affinity Sensors, Inc. Biosensor methods have the advantage of being performed in real time without any requirement for labelling, and are especially suitable for screening large numbers of samples, and consequently they are extremely useful in high-throughput screening of candidate pharmaceutical agents. Recently biosensors have been used extensively for the study of the interactions between peptides and membranes (Mozsolits and Aguilar (in press, 2002).

In particular, sensor chips such as the HPA and L1 chips from BIAcore AB and the Hydrophobic Surface cuvette from Affinity Sensors, Inc are useful in the study of lipid interactions. See for example U.S. Pat. No. 5,922,594 by Lofang, and the papers by Valdez-Gonzalez et al and by Ariga et al. referred to above. However, as far as the inventors are aware there has been no suggestion that a biosensor technique could be used for the high-throughput screening of candidate agents for the treatment of any amyloid-related condition.

We have now surprisingly found that the binding of an amyloid protein to isolated membranes from a given cell type, as measured using a biosensor, correlates very well with the degree of toxicity against that cell type, as measured by a cytotoxicity assay. Thus the invention uses the biosensor to measure binding of amyloid protein to isolated membranes from a cell type such as a neuron, as an index of toxicity. In this way, compounds can be rapidly screened for their therapeutic properties. While it was previously known that Aβ can bind to synthetic lipids or to cells in culture, this is the first demonstration that actual cell membranes can be used for this purpose in a biosensor.

### SUMMARY OF THE INVENTION

In a first aspect, the invention provides a device for screening of candidate agents for treatment of a condition involving cerebral amyloidosis, cerebral angiopathy, or systemic amyloidosis, comprising a biosensor membrane coupled to a lipid preparation which comprises cholesterol and phospholipid.

Preferably the lipid preparation comprises about 30% to 80% cholesterol. More preferably the lipid preparation is a plasma membrane-enriched fraction of a cellular homogenate of smooth muscle cells, nerve cells, kidney cells, cardiac myocytes, or hepatocytes, chosen according to the target condition. For a condition involving a cerebral amyloidosis or cerebral angiopathy, the lipid preparation is prepared from smooth muscle cells or nerve cells. Even more preferably the cells are vascular smooth muscle cells or neuronal cells. For the systemic amyloidoses, the major tissues involved are kidney, heart and liver. Therefore for these conditions the cellular homogenate is prepared from kidney cells, cardiac myocytes or hepatocytes.

The cells may be obtained from primary tissue samples, or may be prepared from cells of a primary or transformed cell line. For neuronal cells, cell lines or primary cells of cortical or hippocampal origin are particularly preferred.

In a second aspect, the invention provides a method of screening of candidate agents for treatment of a condition involving cerebral amyloidosis, cerebral angiopathy, or systemic amyloidosis, comprising the step of assessing the effect of a candidate agent on binding of an amyloid peptide to a lipid preparation which comprises cholesterol and phospholipid, in which inhibition of binding indicates potentially useful activity.

In a third aspect, the invention provides a method of screening of candidate agents for potential toxicity to the brain or to cerebral blood vessels, comprising the step of assessing the effect of a candidate agent on binding of an amyloid peptide to a lipid preparation which comprises cholesterol and phospholipid, in which promotion of binding indicates potentially toxic activity.

Preferably the lipid preparation is coupled to a biosensor membrane. Preferably the lipid preparation comprises about 30% to 80% cholesterol. More preferably the lipid preparation is a plasma membrane-enriched fraction of muscle cells or nerve cells. Even more preferably the cells are vascular smooth muscle cells or neurons.

Preferably where the amyloid peptide is Aβ, the method is performed in the presence of acetylcholinesterase (AChE). More preferably AChE is added to the Aβ prior to its addition to the lipid preparation. Most preferably in this form of the invention the lipid preparation is coupled to a biosensor membrane. Suitably, both AChE and Aβ are at a concentration of up to about 10 μM.

In a fourth aspect the invention provides an agent for the treatment of a condition involving cerebral amyloidosis or cerebral angiopathy, which is a compound which has the ability to inhibit binding of an amyloid peptide to a lipid preparation which comprises cholesterol and phospholipid.

In one preferred embodiment the compound also has the ability to inhibit cholesterol biosynthesis.

In a fifth aspect the invention provides a composition for the treatment of a condition involving cerebral amyloidosis, cerebral angiopathy, or systemic amyloidosis, comprising a compound which has the ability to inhibit binding of an amyloid peptide to a lipid preparation which comprises cholesterol and phospholipid, together with a pharmaceutically acceptable carrier.

In a sixth aspect the invention provides an method of treatment of a condition involving cerebral amyloidosis, cerebral angiopathy, or systemic amyloidosis, comprising the step of administering an effective amount of a compound which has the ability to inhibit binding of an amyloid peptide to a lipid preparation which comprises cholesterol and phospholipid to a subject in need of such treatment.

The amyloid protein will generally be selected from those known to be associated with the target condition. For example, where the condition involves cerebral amyloidosis or cerebral angiopathy, the amyloid peptide will preferably be Aβ. Where the amyloidosis is immunoglobulin light chain-related (AL) amyloidosis, the protein will be immunoglobulin light chain or a biologically functional fragment thereof, preferably the light chain variable region. For secondary amyloidosis the amyloid protein is preferably amyloid A. For familial amyloidosis, the protein is preferably selected from the group consisting of transthyretin, apolipoprotein A-I, gelsolin, fibrinogen A α, and lysozyme.

The condition involving cerebral amyloidosis or cerebral angiopathy may be a sporadic condition such as Alzheimer's disease, amyloidosis associated with Down syndrome, prion-related cerebral amyloidosis, including Creutzfeld-Jacob disease and its new variant associated with “mad cow” disease, or sporadic cerebral angiopathy, or may be a familial condition such as one of the several forms of autosomal dominant forms of familial Alzheimer's disease (reviewed in St George-Hyslop, 2000); hereditary cerebral haemorrhage associated with the Flemish, Arctic, Dutch, or Italian mutations of Aβ precursor protein (reviewed in Ghiso and Frangione, 2001); hereditary cerebral haemorrhage with amyloidosis (Icelandic type); meningocerebrovascular and oculoleptomeningeal amyloidosis; familial British dementia; familial Danish dementia; Cystatin C-related cerebral amyloid angiopathy; transthyretin-related cerebral amyloid angiopathy; and gelsolin-related spinal and cerebral amyloid angiopathy.

Preferably the condition involving cerebral amyloidosis or cerebral angiopathy is sporadic or familial Alzheimer's disease, amyloidosis associated with Down syndrome, sporadic cerebral angiopathy, prion-related cerebral amyloidosis, familial British dementia, Cystatin C-related cerebral amyloid angiopathy, transthyretin-related cerebral amyloid angiopathy, or gelsolin-related spinal and cerebral amyloid angiopathy.

The condition involving systemic amyloidosis may be primary amyloidosis, or may be a reactive amyloidosis or a familial amyloidosis. Preferably this condition is selected from the group consisting of AL, familial transthyretin-associated amyloidosis, amyloid protein A-associated amyloidosis, or familial amyloidosis associated with apolipoprotein A-I, gelsolin, fibrinogen A α, or lysozyme.

It will be clearly understood that the method of treatment and the composition according to the invention may be used in conjunction with other treatments for the relevant condition. For example, where the condition involves cerebral amyloidosis or cerebral angiopathy, particularly Alzheimer's disease, they may be used in conjunction with treatment with another agent such as an acetylcholinesterase active site inhibitor, for example phenserine, galantamine, or tacrine; an antioxidant, such as Vitamin E or Vitamin C; an oestrogenic agent such as 17-β-oestradiol; a chelating agent, such as clioquinol; or AChE peripheral site inhibitor such as propidium or gallamine.

In one embodiment, the condition is associated with an amyloid-type protein other than Aβ, such as synuclein. In this embodiment, the condition is selected from the group consisting of Parkinson's disease, dementia with Lewy body formation, multiple system atrophy, Hallerboden-Spatz disease, and diffuse Lewy body disease.

The term “subject” as used herein refers to any mammal having a disease or condition which requires treatment with a pharmaceutically-active agent. The mammal may be a human, or may be a domestic or companion animal. While it is particularly contemplated that the compounds of the invention are suitable for use in medical treatment of humans, they are also applicable to veterinary treatment, including treatment of companion animals such as dogs and cats, and domestic animals such as horses, cattle and sheep, or zoo animals such as apes and monkeys, felids, canids, bovids, and ungulates.

Methods and pharmaceutical carriers for preparation of pharmaceutical compositions are well known in the art, as set out in textbooks such as Remington's Pharmaceutical Sciences, 20th Edition, Williams & Wilkins, Pennsylvania, USA.

The compounds and compositions of the invention may be administered by any suitable route, and the person skilled in the art will readily be able to determine the most suitable route and dose for the condition to be treated. Dosage will be at the discretion of the attendant physician or veterinarian, and will depend on the nature and state of the condition to be treated, the age and general state of health of the subject to be treated, the route of administration, and any previous treatment which may have been administered. The compound of the invention may optionally be administered in conjunction with one or more other pharmaceutically-active agents suitable for the treatment of the condition, ie it may be given together with, before, or after one or more such agents.

The carrier or diluent, and other excipients, will depend on the route of administration, and again the person skilled in the art will readily be able to determine the most suitable formulation for each particular case.

As used herein, the term “therapeutically effective amount” means an amount of a compound of the present invention effective to yield a desired therapeutic response, for example to prevent or treat a disease which is susceptible to treatment by administration of a pharmaceutically-active agent.

The specific “therapeutically effective amount” will of course vary with such factors as the particular condition being treated, the physical condition and clinical history of the subject, the type of animal being treated, the duration of the treatment, the nature of concurrent therapy (if any), and the specific formulations employed and the structure of the compound or its derivatives.

As used herein, a “pharmaceutical carrier” is a pharmaceutically acceptable solvent, suspending agent, excipient or vehicle for delivering the compound of the invention and/or another pharmaceutically-active agent to the subject. The carrier may be liquid or solid, and is selected with the planned manner of administration in mind.

The compound of the invention may be administered orally, topically, or parenterally in dosage unit formulations containing conventional non-toxic pharmaceutically acceptable carriers, adjuvants, and vehicles. The term parenteral as used herein includes subcutaneous, intravenous, intramuscular, intrathecal, intracranial, injection or infusion techniques.

Generally, the terms “treating”, “treatment” and the like are used herein to mean affecting a subject, tissue or cell to obtain a desired pharmacological and/or physiological effect. The effect may be prophylactic in terms of completely or partially preventing a disease or sign or symptom thereof, and/or may be therapeutic in terms of a partial or complete cure of a disease. “Treating” as used herein covers any treatment of, or prevention of disease in a vertebrate, a mammal, particularly a human, and includes: preventing the disease from occurring in a subject which may be predisposed to the disease, but has not yet been diagnosed as having it; inhibiting the disease, ie., arresting its development; or relieving or ameliorating the effects of the disease, ie., causing regression of the effects of the disease.

The invention includes various pharmaceutical compositions useful for ameliorating disease. The pharmaceutical compositions according to one embodiment of the invention are prepared by bringing a compound of the invention and optionally one or more other pharmaceutically-active agents or combinations of the compound of the invention and one or more other pharmaceutically-active agents into a form suitable for administration to a subject, using carriers, excipients and additives or auxiliaries.

Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 20th ed. Williams & Wilkins (2000) and The British National Formulary 43rd ed. (British Medical Association and Royal Pharmaceutical Society of Great Britain, 2002; http://bnf.rhn.net), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed., 1985).

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

For the purposes of this specification it will be clearly understood that the word “comprising” means “including but not limited to”, and that the word “comprises” has a corresponding meaning.

### BRIEF DESCRIPTION OF THE FIGURES

FIG. 1 shows the effect of Aβ peptides on vascular SMC viability. Vascular SMC cultures were incubated with Aβ peptides (10 μM, unaged or aged for 5 days) for 24 h. The decrease in MTS release was calculated as a percentage of the mean value for cultures in which no peptide was added. Values are means±SEM (n=3). Asterisks show values that are significantly different from the values obtained from the corresponding incubation using unaged peptide (P<0.05, Student's t test).

FIG. 2 shows the levels of amyloid fibrils in fresh or aged Aβ peptide preparations, as measured by a Congo Red binding assay. Aβ peptides were allowed to age for 5 days or prepared fresh and then incubated with Congo Red. The concentration of amyloid fibrils was determined spectrophotometrically. Asterisks show values which are significantly different from the values obtained from the corresponding experiment using unaged peptide (P<0.05, Student's t test).

FIGS. 3A and B illustrates sensorgrams showing the binding of various concentrations of unaged Aβ 1-42 (A) and Aβ 1-40 (B) to SUV containing 60% cholesterol and 40% phospholipid. The binding is shown in response units (RU).

FIG. 3C shows the results of Scatchard plot analysis of the binding of Aβ1-42 and Aβ1-40 to SUV containing 60% cholesterol and 40% phospholipid. R_{eq}=theoretical maximum value of RU for each concentration. C (=concentration of Aβ in μM) was used as an approximation of the total amount of unbound Aβ.

FIG. 4 shows the results of quantitative analysis of the effect of aging on the binding of Aβ peptides to synthetic phospholipid membranes. Aβ peptides (10 μM), aged for 0 or 5 days, were injected over a cholesterol: phospholipid (60:40, w/w) surface. The binding is shown in response units recorded 20 min after addition of the peptide (RU₂₀). Values are means±SEM (n=3). Asterisks show values which are significantly different from the values obtained from the corresponding experiment using unaged peptide (P <0.05, Student's t test).

FIG. 5 shows the effect of the cholesterol: phospholipid ratio on the binding of Aβ 1-40 and 1-42 to a synthetic lipid membrane. Unaged Aβ peptides (10 μM) were injected over the membrane surface on the biosensor, and the binding was measured. The binding is shown in response units recorded 20 min after addition of the peptide (RU ₂₀). Values are means±SEM (n=3). Asterisks show values for Aβ 1-42 which are significantly different from the corresponding incubation using Aβ 1-40 (P<0.05, Student's t test). PL=phospholipid.

FIG. 6 shows the effect of lovastatin on the binding of Aβ peptides to vascular SMC membranes. Unaged Aβ peptides (10 μM) were injected over the lovastatin-treated (10 μg/ml) and untreated vascular SMC membrane surface, and the binding was measured. A. Aβ binding in response units recorded 20 min after addition of the peptide (RU ₂₀). Bars show mean values±SEM (n=3). B. Cholesterol content of the crude plasma membrane preparations from untreated and lovastatin-treated cells. The membrane preparation was diluted 1:100 prior to assay. Bars show mean values±SEM (n=3). Asterisks show values which are significantly different from the corresponding incubations without lovastatin (P<0.05, Student's t test).

FIG. 7 shows the effect of lovastatin on Aβ toxicity. Lovastatin-treated and untreated vascular SMC cultures were incubated with unaged Aβ peptides (10 μM). The decrease in MTS release was calculated as a percentage of the mean value for no addition control cultures. Values are means±SEM (n=3). Asterisks show values which are significantly different from the corresponding incubations without lovastatin (P<0.05, Student's t test).

### DETAILED DESCRIPTION OF THE INVENTION

The invention will now be described in detail by way of reference only to the following non-limiting examples and drawings.

Abbreviations used herein are as follows:

| | |
|---|---|
| AD | Alzheimer's disease |
| APP | β-amyloid protein precursor |
| CAA | cerebral amyloid angiopathy |
| DMEM | Dulbecco's modified Eagle's medium |
| DMPC | dimyristoyl-L-a-phosphatidylcholine |
| DMPE | dimyristoyl-L-a |
| | phosphatidylethanolamine |
| MPG | dimyristoyl-L-α-phosphatidylglycerol DMPS |
| | dimyristoyl-L-α-phosphatidylserine |
| | MTS [3-(4,5-dimethylthiazol-2-yl)-5-(3- |
| | carboxymethoxyphenyl)-2(4-sulfophenyl)-2H- |
| | tetrazolium |
| SMC | smooth muscle cell |
| SUV | small 100 nm unilamellar vesicles. |

We have examined the binding of Aβ peptides both to synthetic lipid bilayers and to plasma membrane-enriched preparations derived from vascular smooth muscle cells (SMCs), using surface plasmon resonance. We have found that the extent of binding of Aβ to membranes correlates very well with the extent of Aβ toxicity. Importantly, we have demonstrated that Aβ binding to synthetic lipids and intact SMC membranes requires the presence of cholesterol, and that reduction of membrane cholesterol content with an inhibitor of cholesterol biosynthesis reduces Aβ toxicity.

Our results strongly support the view that cholesterol-lowering drugs reduce Aβ toxicity by reducing Aβ-membrane binding.

Materials

Aβ peptides were synthesised using manual solid-phase Boc (N-tert-butocarbonyl) amino acid synthesis. Peptides were synthesized using manual solid-phase Boc amino acid chemistry with in situ neutralisation.

Acylations were performed using 5 equivalents of the Boc-protected amino acid, 4.9 equivalents of 2-(1H-benzotriazole-1-yl)-1,1,3,3-tetramethyluronium hexafluorophosphate, 5 equivalents of 1-hydroxybenzotriazole and 7.5 equivalents of diisopropylethylamine in dimethyl formamide. Each acylation was monitored using ninhydrin, and couplings were repeated if necessary. Aβ peptides were cleaved from the resin using anhydrous hydrogen fluoride and p-cresol/p-thiocresol. Hydrogen fluoride was then removed, and the peptide was dissolved in trifluoroacetic acid and precipitated with ether.

Peptide purification was achieved using an acetonitrile/water (0.01% trifluoroacetic acid) gradient on a reversed-phase preparative Zorbax high performance liquid chromatography (HPLC) column heated to 60° C. The purity (>95%) and identity of the peptide was analysed by analytical HPLC, electrospray mass spectrometry and amino acid analysis.

Dulbecco's modified Eagle's Medium (DMEM) and penicillin/streptomycin were purchased from Gibco Life Technologies (Mulgrave, Vic, Australia), and foetal bovine serum (heat inactivated) was purchased from Commonwealth Serum Laboratories (Parkville, Vic., Australia). N-octyl-D-glucopyranoside, dimyristoyl-L-α-phosphatidylcholine (DMPC), dimyristoyl-L-α-phosphatidyl-DL-glycerol (DMPG), dimyristoyl-L-α-phosphatidylethanolamine (DMPE), dimyristoyl-L-α-phosphatidylserine (DMPS) and D-cholesterol were purchased from Sigma (St Louis, Mo., USA). Lovastatin was purchased from Calbiochem (Sydney, NSW, Australia), and activated to its active open-ring form as previously described (Jakobisiak et al., 1991).

Solubilization and Aging of A β peptides

Aβ 1-42, Aβ 1-40, Aβ 1-28, Aβ 17-42 and Aβ 29-42 were dissolved in dimethyl sulfoxide (DMSO) at a concentration of 2 mM. The peptide solutions were then sonicated (42 kHz) for 5 minutes, and centrifuged at 5,000 rpm for 1 minute at room temperature using a Hermle Z160M bench microfuge. Solubilised peptides were immediately snap-frozen and mixed on a vortex mixer for 15 seconds. The peptides were then diluted into DMEM for cell culture experiments, or in 0.02 M sodium phosphate buffer, pH 6.8 for biosensor experiments, to give a final concentration of 10 μM. To “age” Aβ peptides, a process which increases the proportion of fibrillar oligomeric species (Jarrett and Lansbury, 1992), peptides were incubated at 37° C. in a humidified atmosphere of 5% CO₂ for 5 days at a concentration of 100 μM.

Congo Red Assay of Amyloid Fibrils

The concentration of amyloid fibrils was measured using the assay of Klunk et al. (1999). Aβ peptides were mixed with Congo red (CR) in 0.02 M sodium phosphate buffer, pH 6.8. The final concentration of peptide and CR was 10 μM. Solutions of CR alone were also prepared in 0.02 M sodium phosphate buffer, pH 6.8. The mixture was vortexed briefly and then incubated at room temperature for 15 min. The absorbances at 403 and 541 nm were measured using a BioRad SmartSpec 3000 spectrophotometer. Background absorbance values of buffer alone were subtracted from the values obtained from each sample. The concentration of Aβ fibrils in each preparation was then determined using the formula

[A fibrils]=(A₅₄₁ₙₘAβ:CR solution/4780)−(A₄₀₃ₙₘ of Aβ:CR solution/6830)−(A₄₀₃ₙₘ of CR solution/8620)

All preparations were prepared in triplicate, and the assay was conducted independently three times, with similar results being obtained in each experiment.

Vascular Smooth Muscle Cell Culture

Vascular SMCs from aortae of Sprague-Dawley rats were cultured in DMEM supplemented with 10% foetal bovine serum and penicillin/streptomycin at 37° C. in a humidified atmosphere of 5% CO₂. Vascular SMCs were plated at a density of 10⁴ cells/well in a 96-well plate, or at a density of 10⁶ cells/75 cm² cell culture flask (Nunc, Denmark) in 20 ml culture medium, and grown to 80% confluence, after which the medium was removed and replaced with fresh, serum-free medium with or without to μM lovastatin. The cells were then incubated for 72 h. The cells were then either used for the preparation of membranes, or incubated with Aβ peptides (10 μM) for cytotoxicity assay. In the latter case, Aβ peptides were added to the culture medium and the cells incubated for a further 24 h. In control incubations, vehicle alone, ie lacking peptide, was added.

MTS Assay of Cytotoxicity

Cytotoxicity was determined using the CellTiter 96 AQueous One Solution Cell Proliferation Assay kit (Promega Corporation, Madison, Wis., USA) (Cory et al., 1991). The F3-(4,5-dimethylthiazol-2-yl)-5-(3-carboxymethoxyphenyl)-2(4-sulfophenyl)-2H-tetrazolium (MTS) reagent solution was added to the culture medium at a concentration of 10% (by volume). The cells were then incubated for a further 2 h at 37° C., and the absorbance of the samples read at a wavelength of 560 nm using a Wallac Victor 1420 plate reader.

Plasma Membrane Preparation

A crude plasma membrane preparation was prepared from vascular SMCs by differential centrifugation (Hubbard et al., 1983). Cells were scraped off 10×75 cm² flasks using a cell scraper, and centrifuged in DMEM at 3,000 rpm in a Beckman Coulter Allegra 21R centrifuge at 4° C. for 3 min. The pellet was then washed with phosphate-buffered saline (PBS), added to 10 ml of STM buffer (0.25 M sucrose/5 mM Tris-HCl, pH 7.4/1.0 mM MgCl₂), and homogenized on ice using 10 up and down strokes in a 40 ml Dounce-type glass homogenizer with a loose-fitting pestle. The homogenate was centrifuged at 1,100 rpm for 5 min. The supernatant fraction was saved and the pellet rehomogenized in 5 ml of STM buffer. The suspension was again centrifuged, and the first and second supernatant fractions combined, then centrifuged at 40,000 rpm for 2 h (Beckman L8-M Ultracentrifuge, 70 Ti rotor, no brake) and the resulting crude plasma membrane fraction resuspended in 1.0 ml of 0.02 M sodium phosphate buffer, pH 7.4. Total membrane cholesterol was determined using the Amplex Red cholesterol assay kit (Molecular Probes, Eugene, Oreg., USA). The protein content of the membrane preparations was determined using the bicinchoninic acid (BCA) assay using bovine serum albumin as standard.

Preparation of Synthetic Model Membranes

Small 100 nm unilamellar vesicles (SUV), containing DMPC, DMPG, DMPS and DMPE, and cholesterol, were prepared in 0.02 M phosphate buffer (pH 6.8) by sonication and extraction. Briefly, 1.5 mg of total lipid was dissolved in 1.5 ml of CHCl₃: MeOH (3:1, v/v). Aliquots (408 μl) were removed and evaporated under a stream of nitrogen, and the lipids further dried in vacuo overnight. The lipids were then resuspended in 600 μl of 0.02 M sodium phosphate buffer, pH 6.8. The resulting lipid dispersion was sonicated in a bath type sonicator until clear, and then extruded 17 times through 100 nm pore diameter polycarbonate filters using Liposofast apparatus (Avestin, Ottawa, Canada) to obtain 100 nm SWV. The mixed lipid vesicles contained 80% (w/w), 60% (w/w), 40% (w/w), 30% (w/w) or 0% (w/w) cholesterol. The remaining lipid comprised a mixture of DMPC: DMPE: DMPS: DMPG in a ratio of 75:20:2.5:2.5 (by weight).

Binding Studies

Binding experiments were carried out with a BIAcore X analytical system (Biacore, Uppsala, Sweden) using an L1 sensor chip (Biacore), as supplied by the manufacturer. It will be appreciated that other types of biosensor may also be used, preferably provided that the chip surface is such that the bound lipid material is able to retain a bilayer or monolayer structure. The running buffer used for all experiments was 0.02 M sodium phosphate buffer, pH 6.8 (phosphate buffer). The washing solution was 40 mM N-octyl β-D-glucopyranoside. The regeneration solution was 10 mM sodium hydroxide. All solutions were freshly prepared, degassed and filtered through a 0.22 μM filter. The operating temperature was 25° C.

The alkyl surface of the L1 chip was cleaned by an injection of 25 μl of non-ionic 40 mM octyl glucoside at a flow rate of 5 μl/min. SUV (100 μl) or vascular SMC membranes (100 μl containing 0.33 mg protein) were then immediately applied to the chip surface at a low rate of 5 μl/min. To remove any multilamellar structures from the synthetic lipid surface, 30 μL of 10 mM sodium hydroxide was injected at a flow rate of 50 gl/min, resulting in a stable baseline corresponding to the successful formation of an immobilized layer of SUVs.

Peptide solutions were prepared at concentrations ranging from 0.5 to 10 μM. The solutions were injected over the lipid surface at a flow rate of 5 μl/min for 20 min. The peptide solution was then replaced by phosphate buffer and the peptide-membrane complex allowed to dissociate. The removal of the bound peptide and regeneration of the L1 chip surface, without removal of the synthetic lipid or vascular SMC membrane layer, was achieved by an injection of sodium hydroxide (30 μl, 10 mM) at a flow rate of 50 μl/min.

The amount of binding in response units (RU) was fitted by a simple one-to-one Langmuir reaction model (Morton et al., 1995):

A+B⇄AB

or a competing reaction model (Karlsson and Falt, 1997):

A+B⇄AB and A+C⇄AC

using BIAevaluation 3.0 software (Biacore). The data obtained from five different binding experiments performed at five different concentrations of peptide were fitted globally to obtain the equilibrium association constant (K_{A}). The accuracy of each fit was assessed by calculating a X² value for the data (Karlsson and Failt, 1997). A lower X² value was taken as an indication of a better fit of the data. The binding was found to approach equilibrium after incubation with the peptide for 20 min. Therefore for routine quantification of total binding to the membrane, the RU value obtained 20 min after addition of the peptide (RU₂₀) was taken as an estimate of maximum binding at each concentration (R_{eq}). Scatchard plots were generated using BIAevaluation 3.0 software (Biacore).

### EXAMPLE 1

Toxicity of Aβ Against Vascular Smooth Muscle Cells

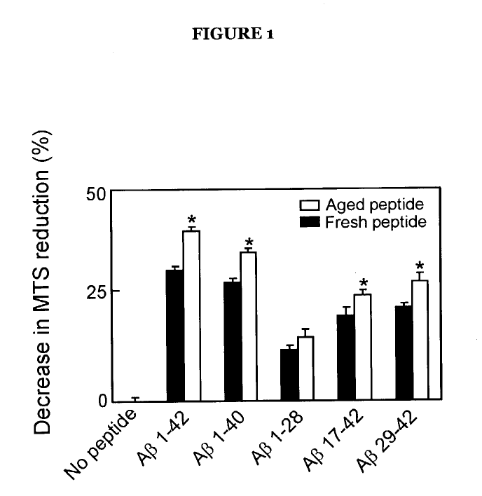

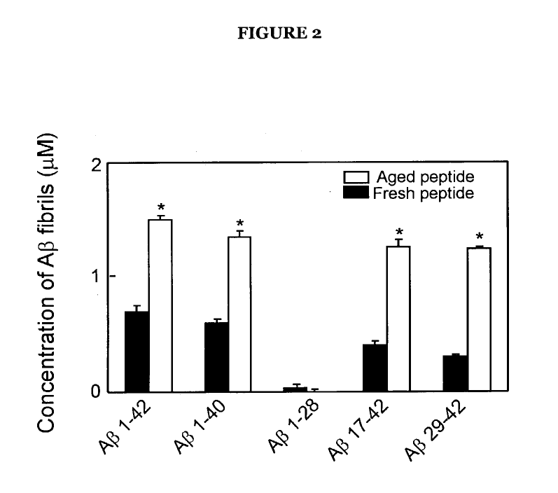

To measure Aβ toxicity against vascular SMCs, cultures of cells were treated with Aβ peptides and analogues (10 μM) and the amount of toxicity determined using the MTS assay (Cory et al., 1991). Aβ1-40, Aβ1-42, Aβ29-42 and Aβ17-42 were all found to cause significant toxicity, as shown in FIG. 1. Aβ1-28 was less toxic, suggesting that the toxicity of Aβ may be associated with the more hydrophobic C-terminal region of the peptide. Aging the peptides by incubation for 5 days caused a significant increase in toxicity. Previous studies (e.g., Pike et al., 1991) have shown that the aggregation of the Aβ into fibrils may be important for the generation of toxic species. The present results support this conclusion. Incubation of Aβ peptides for 5 days significantly increased the proportion of fibrillar species as determined by a Congo red binding assay, as shown in FIG. 2. In general, the amount of Aβ toxicity approximately correlated with the proportion of fibrillar species. For example, there was a significant increase in cytotoxicity after aging Aβ 1-42 (p<0.05; Student's t test). In addition, aged Aβ 1-28, which formed fewer fibrillar species than the other Aβ species tested, was significantly less toxic to vascular SMC cultures.

### EXAMPLE 2

Binding of Aβ Peptides to Lipids

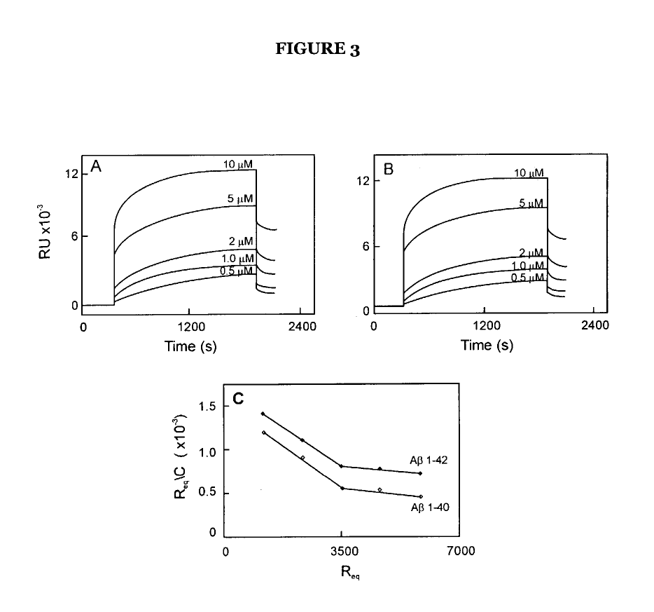

As previous studies have shown that Aβ can interact directly with lipid membranes, we examined the possibility that the toxic effects of Aβ were due to a direct interaction with the vascular SMC membrane. To study the binding of Aβ to lipids, biosensor technology was employed. Initially a biosensor chip was coated with a synthetic lipid mixture containing 60% cholesterol, 30% DMPC, 8% DMPE, 1% DMPS and 1% DMPG, and sensorgrams obtained for Aβ 1-42 and Aβ 1-40. These are shown in FIG. 3A and FIG. 3B respectively. The peptides bound to the lipid surface in a biphasic manner. The initial association of the peptide to the lipid surface was rapid. Maximum binding was approached approximately 20 min after application of the peptide. The dissociation curve of the bound complex followed a similar biphasic pattern. The signal fell rapidly at the end of the injection period, since the peptide was no longer present and the buffer flow removed a large amount of weakly bound peptide. Typically, the peptide sensorgrams did not return to zero until the biosensor was stripped with 10 mM NaOH, indicating that a proportion of the peptide remained bound to the surface.

The biphasic nature of the AB-lipid interaction was confirmed by a curve fitting analysis of the association and dissociation curves. A poor fit was obtained using the simplest one to one Langmuir binding model (Morton et al., 1995) (X² for Aβ1-42=1035; X² for Aβ1-40=1200). However, a significantly improved fit was obtained using a competing reaction model (Karlsson and Falt, 1997) (X² for Aβ1-42=400; X² for Aβ1-40=370). Scatchard plot analysis of the binding data was also consistent with a biphasic interaction for both Aβ1-42 and Aβ1-40, as shown in FIG. 3C.

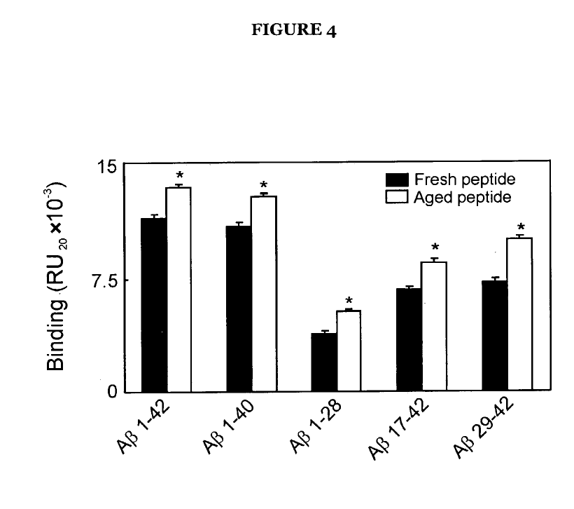

A comparison of Aβ peptides and analogues for their abilities to bind to the synthetic lipid mixture showed that there was good agreement between the extent of lipid binding, as shown in FIG. 4, and the amount of toxicity caused by each peptide as shown in FIG. 1. For example, the more toxic C-terminal Aβ fragments (Aβ 29-42 and Aβ 17-42) bound more strongly than the less toxic N-terminal fragment (Aβ 1-28).

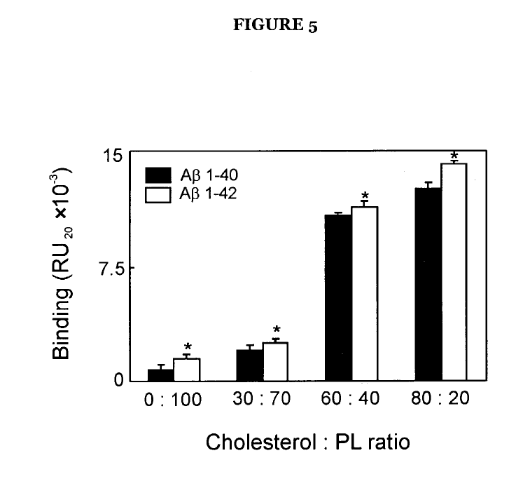

The ratio of cholesterol to phospholipid in the synthetic lipid mixture was directly related to the extent of high-affinity lipid binding, as illustrated in FIG. 5. When a pure phospholipid mixture was used, very little binding was observed. However, at higher concentrations between 30-80% cholesterol, the amount of binding increased. This increase in binding was also reflected by an increase in the equilibrium association constants determined after fitting the kinetic data from FIGS. 3A and 3B to a competing reaction model. As shown in Table 1, both Aβ 1-42 and Aβ 1-40 peptides had a higher binding affinity for synthetic lipid mixtures containing a greater percentage of cholesterol.

### Table 1

Values of equilibrium association constants (K_{A}) for the binding of Aβ to synthetic lipid mixtures based on a competing reaction model of binding

| Cholesterol: PL ratio | Peptide | K_{A1} | K_{A2} |
|---|---|---|---|
| 30 : 70 | Aβ1-42 | 1.8 × 10⁵ | 6.9 × 10⁴ |
| | Aβ1-40 | 1.1 × 10⁵ | 5.2 × 10⁴ |
| 60 : 40 | Aβ1-42 | 2.0 × 10⁸ | 4.8 × 10⁷ |
| | Aβ1-40 | 4.0 × 10⁷ | 1.3 × 10⁶ |
| 80 : 20 | Aβ1-42 | 2.7 × 10⁸ | 6.9 × 10⁷ |
| | Aβ1-40 | 4.8 × 10⁷ | 1.0 × 10⁷ |

K_{A1} = equilibrium association constant for high affinity binding component.
K_{A2} = equilibrium association constant of low-affinity binding component.

### EXAMPLE 4

Effect of Cholesterol on Binding

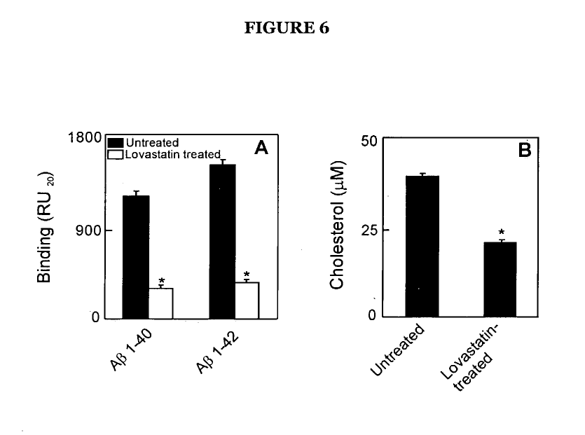

To determine whether the cholesterol content of the vascular SMC membrane influences the binding of Aβ, we prepared a plasma membrane-enriched fraction from vascular smooth muscle cells and applied this fraction to the biosensor chip. When applied at a concentration of 10 μM, both Aβ1-40 and Aβ1-42 bound to the membrane fraction, as shown in FIG. 6. The total amount of binding was approximately 10% of that observed for a synthetic lipid mixture containing 60% cholesterol and 40% phospholipid, as shown in FIG. 5.

### EXAMPLE 5

Effect of a Cholesterol Synthesis Inhibitor on Binding

The effect of lovastatin, a cholesterol biosynthesis inhibitor, on Aβ binding to the membrane was also examined. Cells were pretreated with lovastatin for 72 h and then a plasma membrane-enriched fraction was prepared. The protein composition of the membrane fractions was closely similar in the control (3.29±0.15 mg/ml) and lovastatin-treated groups (3.24±0.03 mg/ml), indicating that lovastatin treatment did not significantly alter the amount of total membrane protein. Treatment of the cells with lovastatin was found to strongly decrease binding of both Aβ1-40 and Aβ1-42 to the membrane fraction, as shown in FIGS. 6A and B). After lovastatin treatment, the cholesterol content of the membrane fraction was reduced to approximately 55% of that recovered from untreated cells (FIG. 6, panel B). Aβ1-40 and Aβ1-42 binding to the plasma membrane fraction of lovastatin-treated cells was approximately 25% of that achieved without lovastatin pretreatment. As indicated by their equilibrium association constants, both peptides displayed a higher binding affinity for untreated vascular SMC membranes than for membranes treated with lovastatin. This is summarised in Table 2.

TABLE 2
Values of equilibrium association constants for
the binding of Aβ to vascular SMC membranes
based on a competing reaction model of binding

| Treatment group | Peptide | K_{A1} | K_{A2} |
|---|---|---|---|
| Untreated | Aβ1-42 | 1.8 × 10⁶ | 9.6 × 10⁵ |
| | Aβ1-40 | 8.3 × 10⁵ | 5.9 × 10⁵ |
| Lovastatin- | Aβ1-42 | 9.3 × 10⁴ | 5.3 × 10⁴ |
| Treated | Aβ1-40 | 4.1 × 10⁴ | 3.6 × 10⁴ |

K_{A1} = equilibrium association constant for high affinity binding component.
K_{A2} = equilibrium association constant of low-affinity binding component.

### EXAMPLE 6

Effect of Inhibiting Cholesterol Synthesis on Toxicity

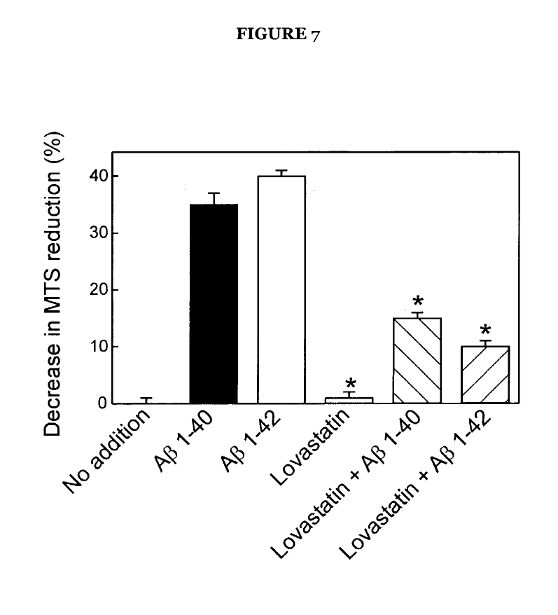

To examine whether this decrease in binding might have any consequences for Aβ toxicity, the amount of Aβ toxicity was measured following lovastatin treatment of cells, using the MTS assay. The results are shown in FIG. 7. Lovastatin alone had little effect on the ability of vascular SMCs to reduce MTS. However, cells pretreated with lovastatin were more resistant than untreated cells to Aβ toxicity, as the Aβ1-40 and Aβ1-42-induced decrease in MTS reduction was approximately 25-40% lower in lovastatin-treated cells than in controls.

Discussion

We have demonstrated that cholesterol is required for the binding of Aβ to synthetic lipid mixtures and to vascular SMC membranes. Our results also suggest that lowering plasma membrane cholesterol can decrease Aβ toxicity. Taken together, our results indicate that binding of Aβ to the lipid component of the plasma membrane is required for Aβ toxicity.

Analysis of the sensorgram data indicated that the binding of Aβ to lipid membranes is more complicated than a simple one to one interaction. This conclusion was also supported by the Scatchard plots, which were nonlinear (i.e., biphasic). While several interpretations of these data are possible, one possibility is that Aβ exists in multiple states, each of which binds to lipids with a different affinity. Aging the peptides by incubation for days was found to increase Aβ binding and to increase the concentration of amyloid fibrils. Unaged Aβ also contained some amyloid fibrils, suggesting that different oligomeric species have different affinities for lipid binding. For this reason, the data from Scatchard analysis must be interpreted with caution. The binding may be biphasic; however, more data may reveal a more complex interaction. For the same reason, affinity constants calculated from the competing reaction model, as reported in Tables 1 and 2, should be viewed as indicating an overall affinity of Aβ for the lipid membrane, rather than as having any independent significance.

There have been conflicting reports on the role of cholesterol in Aβ toxicity. Zhou and Richardson (1996) reported that methyl-β-cyclodextrin-cholesterol protects cells from Aβ-mediated toxicity. However, in this study, cholesterol was added exogenously, and the lipid composition of the plasma membrane was not analyzed. In contrast, Wang et al. (2001) reported that methyl-β-cyclodextrin alone attenuated Aβ toxicity by lowering cell cholesterol, and suggested that the effect of methyl-β-cyclodextrin-cholesterol might be related more to a loss of cellular cholesterol, rather than to the addition of exogenous cholesterol. Our own studies strongly support this view, as well as providing a biochemical explanation for the decreased toxicity.

There is good evidence that Aβ-membrane binding is involved in Aβ toxicity. Aβ can bind to phospholipids (Waschuk et al., 2001), and inhibition of electrostatic interactions between Aβ and the negative phospholipids can inhibit Aβ toxicity (Hertel et al., 1997). We have found that bound Aβ was easily removed from lipid membranes with sodium hydroxide, suggesting that electrostatic, rather than hydrophobic forces, are involved. This contrasts with the behaviour of many other peptides, which bind hydrophobically and penetrate deeply into the lipid bilayer (Mozsolits et al., 2001). Indeed, although cholesterol can bind Aβ, presumably via hydrophobic interactions, Ji et al. have shown that Aβ-cholesterol binding inhibits fibril formation, and have speculated that cholesterol might be neuroprotective. However, in our studies, increased cholesterol was clearly associated with increased toxicity. Therefore direct binding of Aβ to cholesterol may not be involved in the Aβ-membrane interaction in our system. Aβ can also bind to sphingolipids and gangliosides (Ariga et al., 2001; Valdez-Gonzalez et al., 2001; Mahouf et al., 200). Thus it is possible that cholesterol plays some other role, perhaps in the control of membrane fluidity. This concept fits in well with our finding that changing the cholesterol content changes the equilibrium association constant (K_{A}) for binding, i.e. that the properties of the entire membrane are altered by cholesterol to increase the affinity of binding.

The mechanism by which Aβ binding to the cell membrane causes cell toxicity is still unclear. Free radical production, lipid peroxidation and alterations in ion channel function have been implicated (reviewed by Small and McLean, 1999). If Aβ binds directly to the lipid component of the membrane, then alterations in membrane fluidity may occur. Chochina et al. (2001), using synaptic plasma membranes, have reported that Aβ 1-40 can increase neuronal membrane fluidity, although in contrast to this Kremer et al. (2001), using synthetic lipids, found that Aβ can decrease membrane fluidity. Certainly changes in membrane fluidity could affect the function of a variety of proteins on the cell surface, including ion channels. For example, alterations in fluidity are known to affect the sub-membrane localization and function of the nicotinic receptor (Baenziger et al., 2000).

We have found that the extent of Aβ aggregation correlates with the vascular SMC toxic response. Although the process of aging increases the number of amyloid fibrils formed from Aβ, as assessed using a CR binding assay, this does not prove that fibrils are the major toxic form of Aβ. For example, even though aged Aβ 1-28 failed to form fibrils, it was still toxic to vascular SM, albeit in a significantly less pronounced manner than the other peptides. Aβ is probably secreted as a monome, and subsequently aggregates into soluble oligomers or fibrils (Podlisny et al., 1998). It is likely that the levels of soluble oligomeric species of Aβ are also increased by the process of aging. A recent study by Lambert et al. (1998) found that small, low molecular weight oligomers of Aβ 1-42 are several orders of magnitude more potent as neurotoxins than high molecular weight fibrillar species of Aβ 1-42.

Therefore more work is needed to define the precise nature of the toxic form of Aβ, and to ascertain the mechanism of toxicity.

There is some epidemiological evidence that lowering blood cholesterol levels may be of benefit for Alzheimer's disease. In at least one population-based study, the incidence of AD has been found to be higher in individuals with higher cholesterol levels (Roher et al., 1999). The ε4 allele of the apolipoprotein E gene is known to be a risk factor for Alzheimer's disease (Bales et al., 1997; Corder et al., 1993), and demented individuals who are homozygous for the ε4 allele have been reported to have higher plasma cholesterol levels than do normal elderly controls (Czech et al., 1994). This idea is also supported by the observation that AD-like pathology is less severe in APP transgenic mice which have been treated with a cholesterol-lowering drug (Refolo et al., 2001). Indeed, two retrospective studies using cholesterol-lowering statins have reported dramatic decreases in the risk of developing AD (Jick et al., 2000; Wolozin et al., 2000).

Cholesterol could have more than one role in the pathogenesis of AD. A number of studies have shown that cholesterol is also important for the regulation of Aβ production (Mizuno et al., 1998). High cholesterol uptake can increase Aβ deposition in transgenic mice (Refolo et al., 2000; Sparks et al., 1994). Cholesterol depletion can inhibit the generation of Aβ in hippocampal neurons (Simons et al., 1998), and this effect may be mediated by APP secretases (Kojiro et al., 2001). Thus it is possible that inhibition of cholesterol biosynthesis as a therapeutic strategy for AD may have a dual beneficial role, not only decreasing AD production in the brain, but also decreasing the toxic consequences of Aβ accumulation. As cholesterol biosynthesis inhibitors have few major toxic side effects, this approach, coupled with other therapeutic strategies such as the use of secretase inhibitors (Nunan and Small, 2000) Aβ immunization (Schenk et al., 1999) or acetylcholine esterase inhibitors such as galantamine Reminyl® could become the treatment of choice.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

References cited herein are listed on the following pages, and are incorporated herein by this reference.

References

Alvarez, A., Alarcoón, R., Opaza, C., Campos, E.O., Muñoz, F.J., Calderón, F. H., Dajas, F., Gentry, M. K., Doctor, B. P., De Mello, F., Inestrosa, N. C. (1998) Stable complexes involving acetylcholinesterase and amyloid-β peptide change the biochemical properties of the enzyme and increase the neurotoxicity of alzheimer's fibrils. The Journal of Neuroscience, 18(9):3213-3223

Ariga, T., Kobayashi, K., Hasegawa, A., Kiso, M., Ishida, H., and Miyatake, T. (2001) Characterization of high-affinity binding between gangliosides and amyloid β-protein. Arch. Biochem. Biophys. 388, 225-230

Avdulov, N. A., Chochina, S. V., Igbavboa, U., O'Hare, E. O., Schroeder, F., Cleary, J. P., and Wood, W. G. (1997) Lipid binding to amyloid β-peptide aggregates: preferential binding of cholesterol a J. Neurochem. 68, 2086-2091

Baenziger, J. E., Morris, M L., Darsaut, T. E., and Ryan, S. E. (2000) Effect of membrane lipid composition on the conformational equilibria of the nicotinic acetylcholine receptor. J. Biol. Chem. 275, 777-784

Bales, K. R., Verina, T., Dodel, R. C., Du, Y., Altstiel, L., Bender M., Hyslop, P., Johnstone E. M., Cummins, D. J., Piccardo, P., Ghetti, B., and Paul, S. M. (1997) Lack of apolipoprotein E dramatically reduces amyloid β-peptide deposition. Nat. Genet. 17,263-264

Chochina, S. V., Avdulov, N. A., Igbavboa, U., Cleary, J. P., O'Hare, E. O., and Wood, W. G. (2001) Amyloid β-peptide (1-40) increases neuronal membrane fluidity. Role of cholesterol and brain region. J. Lipid. Res. 42,1292-1297

Corder, E. H., Saunders, A. M., Strittmatter, W. J., Schmechel, D. E., Gaskell, P. C., Small, G. W., Haines, J. L., and Pericak-Vance, M. A. (1993) Gene dose of apolipoprotein E type 4 allele and the risk of Alzheimer's disease in the late onset familial disease. Science 261, 921-923

Cory, A. H., Owen, T. C., Barltrop, J. A., and Cory, J. G. (1991) Use of an aqueous soluble tetrazolium assay for cell-growth assays in culture. Cancer Commun. 3,207-212 Curtain, C. C., Ali, F., Volitakis, I., Cherny, R. A., Norton, R. S., Beyreuther, K., Barrow, C. J., Masters, C. L., Bush, A. I., and Barnham, K. J. (2001) Alzheimer's disease amyloid β binds copper and zinc to generate an allosterically ordered membrane-penetrating structure containing superoxide dismutase-like subunits. J. Biol. Chem. 276, 20466-20473

Czech, C., Forstl, H., Hentschel, F., Monning, U., Besthorn, C., Geigerkabisch, C., Sattel, H., Masters, C., and Beyruether, K. (1994) Apolipoprotein E-4 gene dose in clinically disgnosed Alzhiemer's disease: prevalence, plasma cholesterol levels and cerebrovascular change. Eur. Arch. Psychiatry Clin. Neurosci. 243, 291-292

De Ferrari, G. V., Canales, M. A., Shin, I., Weiner, L. M., Silman, I., Inestrosa, N. C. (2001) A structural motif or acetylcholinesterase that promotes amyloid beta-peptide fibril formation. Biochemistry 40(35):10447-57

Eckert, G. P., Cairns, N. J., Maras, A., Gattaz, W. F., and Muller, W. E. (2000) Cholesterol modulates the membrane-disordering effects of β-amyloid peptides in the hippocampus specific changes in Alzheimer's disease. Dement. Geriatr. Cogn. Disord. 11, 181-186

Falk, R. H., Comenzo, R. L., Skinner, M. (1997) The systemic amyloidosis. The New England Journal of Medicine 337:13 898-909

Fassbender, K., Simons, M., Bergmann, C., Stroick, M., Lutjohann, D., Keller. P., Runz, H., Kuhl, S., Bertsch, T., von Bergmann. K., Hennerici, M., Beyreuther, K., and Hartmann, T. (2001) Simvastatin strongly reduces levels of Alzheimer's disease β-amyloid peptides Aβ 42 and Aβ 40 in vitro and in vivo. Proc. Natl. Acad. Sci. USA. 98, 5856-5861 Frears, E. R., Stephens, D. J., Walters, C. E., Davies, H., and Austen, B. M. (1999) The role of cholesterol in the biosynthesis of β-amyloid. NeuroReport 10, 1699-1705

Friedhoff, L. T., Cullen, E. I., Geoghagen, N. S., and Buxbaum, J. D. (2001) Treatment with controlled-release lovastatin decreases serum concentrations of human β-amyloid (Aβ) peptide. Int. J. Neuropsychopharmacol. 4,127-130

Glenner, G. G., and Wong, C. W. (1984) Alzheimers disease: initial report of the purification and characterization of a novel cerebrovascular amyloid protein. Biochem. Biophys. Res. Commun. 120, 885-890

Hartmann, T. (2001) Cholesterol, Aβ and Alzheimer's disease. Trends Neurosci. 24,S45-S48

Hertel, C., Terzi, E., Hauser, N., Jakob-Rotne, R., Seelig, J., and Kemp, J. A. (1997) Inhibition of the electrostatic interaction between β-amyloid peptide and membranes prevents β-amyloid-induced toxicity. Proc. Natl. Acad. Sci. USA. 94, 9412-9416

Hubbard, A. L., Wall, D. A., and Ma, A. (1983) Isolation of rat hepatocyte plasma membranes. I. Presence of three major domains. J. Cell Biol. 96, 217-229

Inestrosa, N. C. Alvarez, A., Perez, C. A., Moreno, R. D., Vicente, M., Linker, C., Casaneuva, O. I., Soto, C., Garrido, J. (1996) Acetylcholinesterase accelerates assembly of amyloid-beta-peptides into Alzheimer's fibrils: possible role of the peripheral site of the enzyme. Neuron 16(4):881-91

Jakobisiak, M., Bruno, S., Skierski, J. S., and Darzynliewicz, Z. (1991) Cell cycle specific effects of lovastatin. Proc. Natl. Acad. Sci. USA. 88, 3628-3632

Jarrett, J. T., and Lansbury, P. T. Jr. (1993) Seeding “one-dimensional crystallization” of amyloid: a pathogenic mechanism in Alzheimer's disease and scrapie? Cell 73, 1055-1058

Ji, S. R., Wu, Y., and Sui, S. F. (2002) Cholesterol is an important factor affecting the membrane insertion of β-amyloid peptide (Aβ 1-40), which may potentially inhibit the fibril formation. J. Biol. Chem. 277,6273-6279

Jick, H., Zornberg, G. L., Jick, S. S., and Drachman, D. A. (2000) Statins and the risk of dementia. Lancet 356, 1627-1631 Kang, J., Lemaire, H. G., Unterbeck, A., Salbaum, J. M., Masters, C. L., Grzeschik, K. H., Multhaup, G., Beyreuther, K., and Miller-Hill, B. (1987) The precursor of Alzheimer's disease amyloid A₄ protein resembles a cell-surface receptor. Nature 325, 733-736

Karlsson, R. and Falt, A. (1997) Experimental design for kinetic analysis of protein-protein interactions with surface plasmon resonance biosensors. J. Immunol. Methods 200,121-133

Kiunk, W. E., Jacob, R. F., and Mason, R. P. (1999) Quantifying amyloid β-peptide (Aβ) aggregation using the Congo red-Aβ (CR-Aβ) spectrophotometric assay. Analyt. Biochem. 266, 66-76.

Kojro, E., Gimpl, G., Lammick, S., Marz, W., and Fahrenholz, F. (2001) Low cholesterol stimulates the nonamyloidogenic pathway by its effect on the α-secretase ADAM 10. Proc. Natl. Acad. Sci. U.SA. 98, 5815-5820

Kremer, J. J., Sklansky, D. J., and Murphy, R. M. (2001) Profile of changes in lipid bilayer structure caused by β-amyloid pepide. Biochemistry 40, 8563-8471

Kremer, J. J., Pallitto, M. M., Sklansky, D. J., and Murphy, R. M. (2000) Correlation of β-amyloid aggregate size and hydrophobicity with decreased bilayer fluidity in membranes. Biochemistry 39, 10309-10318

Lambert, M. P., Barlow, A. K., Chromy B. A., Edwards, C., Freed, R., Liosatos, M., Morgan, T. E., Rozovsky, I., Trommer, B., Viola, K. L., Wals, P., Zhang, C., Finch, C. E., Krafft, G. A., and Klein, W. L. (1998) Diffusable, nonfibrillar ligands derived from Aβ 1-42 are potent central nervous system neurotoxins. Proc. Natl. Acad. Sci. USA 95, 6448-6453

Mahfoud, R., Garmy, N., Maresca, M., Yahi, N., Puigserver, A, and Fantini, J. (2002) Identification of a common sphingolipid-binding domain in Alzheimer, Prion, and HIV-1 proteins. J. Biol. Chem. 277,11292-11296

Masters, C. L., Simms, G., Weinman, N. A., Multahup, G., McDonald, B. L., and Beyreuther, K. (1985) Amyloid plaque core protein in Alzheimer disease and Down syndrome. Proc. Natl. Acad. Sci. USA 82,4245-4249

Mizuno, T., Haass, C., Michikawa, M., and Yanagisawa, K. (1998) Cholesterol dependant generation of a unique amyloid β-protein from apically missorted amyloid precursor protein in MDCK cells. Biochim. Biophys. Acta. 1373, 119-130

Morton, T. A., Miszka, D. G., and Chaiken I. M. (1995) Interpreting complex binding kinetics from optical biosensors: a comparison of analysis by linearization, the integrated rate equation, and numerical integration. Anal. Biochem. 227,176-185

Morton, T. A. and Miszka, D. G. (1998) Kinetic analysis of macromolecular interactions using surface plasmon resonance biosensors. Meth. Enymol. 295, 268-94

Mozsolits, H. and Aguilar, M.-I. (2002) Surface plasmon resonance spectroscopy: an emerging tool for the study of peptide-membrane interactions Biopolymers (Peptide Science) In Press

Mozsolits, H., Wirth, H. J., Werkmeister, J., and Aguilar, M. I. (2001) Analysis of antimicrobial peptide interactions with hybrid bilayer membrane systems using surface plasmon resonance. Biochim. Biophys. Acta 1512, 64-76

Nunan, J., and Small, D. H. (2000) Regulation of APP cleavage by α-, β- and γ-secretases. FEBS Lett. 483, 6-10

Pike, C. J., Walencewicz, A. J., Glabe, C. G., and Cotman, C. W. (1991) In vitro aging of β-amyloid protein causes peptide aggregation and neurotoxicity. Brain Res. 563, 311-314

Podlinsny, M. B., Walsh, D. M., Amarante, P., Ostaszewski, B. L., Stimson, E. R., Maggio, J. E., Teplow, D. B., and Selkoe D. J. (1998) Oligomizeration of endogenous and synthetic amyloid β-protein at nanomolar levels in cell culture and stabilization of monomer by Congo Red. Biochemistry 37, 3602-3611

Price, J. L., Davis, P. B., Morris, J. C., and White, D. L. (1991) The distribution of tangles, plaques and related immunohistochemical markers in healthy aging ()Alzheimer's disease. Neurobiol. Aging 12, 295-312

Refolo, L. M., Pappolla, J., Malester, M. A., LaFrancois B., Thomas-Bryant, T., Wang, R., Tint, G. S., Sambamurti, K., and Duff, K. E. (2000) Hypercholesterolemia accelerates the Alzheimer's amyloid pathology in a transgenic mouse model. Neurobiol. Dis. 7, 321-331

Refolo, L. M., Pappolla, M. A., LaFrancois, J., Malester, B., Schmidt, S. D., Thomas-Bryant, T., Tint, G. S., Wang, R., Mereken, M., Petanceska, S. S., and Duff, K. E. (2001) A cholesterol-lowering drug reduces β-amyloid pathology in a transgenic mouse model of Alzheimer's disease. Neurobiol. Dis. 8, 890-899

Roher, A. E., Kuo, Y. M., Kokjohn, K. M., Emrmerling, M. R., and Gracon, S. (1999) Amyloid and lipids in the pathology of Alzheimer's disease. Amyloid 6, 136-145

Schenk, D., Barbour, R., Dunn, W., Gordon, G., Grajeda, H., Guido, T., Hu, K., Huang, J., Johnson-Wood, K., Khan, K., Kholodenko, D., Lee, M., Liao, Z., Lieberburg, I., Motter, R., Mutter, L., Soriano, F., Shopp, G., Vasquez, N., Vandervert, C., Walker, S., Wogulis, M., Yednock, T., Games, D., and Seubert, P. (1999) Immunization with amyloid-β attenuates Alzheimer's disease like pathology in the PDAPP mouse. Nature 400, 173-177

Scheuner, D., Eckman, C., Jensen, M., Song, X., Citron, M., Suzuki, N., Bird, T. D., Hardy, J., Hutton, M., Kukull, W., Larson, E., Levy-Lahad, E., Viitanen, M., Peskind, E., Poorkaj, P., Schellenberg, G., Tanzi, R., Wasco, W., Lannfelt, L., Selkoe, D., and Younkin, S. (1996) Secreted amyloid β-protein similar to that in the senile plaques of Alzheimer's disease is incorporated in vivo by the presenilin 1 and 2 and APP mutations linked to familial Alzheimer's disease. Nature Med. 2, 864-870

Shin, I., Silman, I., Weiner, L. M. (1996) Interaction of partially unfolded forms of Torpedo acetylcholinesterase with liposomes. Protein Sci 5(1):42-51

Simons, M., Keller, P., De Strooper, B., Beyreuther, K., Dotti, C., and Simons, K. (1998) Simvastatin strongly reduces levels of Alzheimer's disease β-amyloid peptides Aβ 42 and Aβ 40 in vitro and in vivo. Proc. Natl. Acad. Sci. USA. 95,460-6464

Sparks, D. L., Scheff, S. W., Hunsaker, J. C., Liu, H., and Gross, D. R. (1994) Induction of Alzheimer like β amyloid immunoreactivity in the brains of rabbits with dietary cholesterol. Exp. Neurol. 126, 88-94

Small, D. H., and McLean, C. A. (1999) Alzheimer's disease and the amyloid β protein: What is the role of amyloid? J. Neurochem. 73, 443-449

Small, D. H., Mok, S. S., and Bornstein, J. C. (2001) Alzheimer's disease and Aβ toxicity: from top to bottom. Nature Rev. Neurosci. 2, 595-598

St George-Hyslop, P. H. (2000) Molecular genetics of Alzheimer's disease Biol. Psychiatry 47,183-199

Vinters, H. V. (1987) Cerebral amyloid angiopathy: A critical review. Stroke 18, 311-324

Wang, S. S. S., Rymer, D. L., and Good, T. A. (2001) Reduction in cholesterol and sialic acid content protects cells from the toxic effects of β-amyloid peptides. J.Biol. Chem. 276,42027-42034

Waschuk, S. A., Elton, E. A., Darabie, A. A., Fraser, P. E., and McLaurin, J. A. (2001) Cellular membrane composition defines Aβ lipid interactions. J. Biol. Chem. 276,33561-33568

Wolozin, B., Kellman, W., Ruosseau, P., Celesia, G. G., and Siegel, G. (2000) Decreased prevalence of Alzheimer disease associated with 3-hydroxy-3-methylglutaryl coenzyme A reductase inhibitors. Arch. Neurol. 57,1439-1443

Zhou, Y., and Richardson, J. S. (1996) Cholesterol protects PC12 cells from β amyloid induced calcium disordering and cytotoxicity. Neuroreport 7, 2487-2490

1. A device for screening of candidate agents for treatment of a condition involving cerebral amyloidosis, cerebral angiopathy, or systemic amyloidosis, comprising a biosensor membrane coupled to a lipid preparation which comprises cholesterol and phospholipid.

2. A device according to claim 1, in which the lipid preparation comprises about 30% to 80% cholesterol.

3. A device according to claim 1, in which the lipid preparation is a plasma membrane-enriched fraction of a cellular homogenate of smooth muscle cells, nerve cells, kidney cells, cardiac myocytes, or hepatocytes, chosen according to the target condition.

4. A device according to claim 1, in which the condition involves a cerebral amyloidosis or cerebral angiopathy, and the lipid preparation is prepared from smooth muscle cells or nerve cells.

5. A device according to claim 1, in which the condition is systemic amyloidosis, and the cellular homogenate is prepared from kidney cells, cardiac myocytes or hepatocytes.

6. A method of screening of candidate agents for treatment of a condition involving cerebral amyloidosis, cerebral angiopathy, or systemic amyloidosis, comprising the step of assessing the effect of a candidate agent on binding of an amyloid peptide to a lipid preparation which comprises cholesterol and phospholipid, in which inhibition of binding indicates potentially useful activity.

7. A method of screening of candidate agents for potential toxicity to the brain or to cerebral blood vessels, comprising the step of assessing the effect of a candidate agent on binding of an amyloid peptide to a lipid preparation which comprises cholesterol and phospholipid, in which promotion of binding indicates potentially toxic activity.

8. A method according to claim 6 in which the lipid preparation is coupled to a biosensor membrane.

9. A method according to claim 8, in which the lipid preparation comprises about 30% to 80% cholesterol.

10. A method according to claim 8, in which the lipid preparation is a plasma membrane-enriched fraction of muscle cells or nerve cells.

11. A method according to claim lo, in which the cells are vascular smooth muscle cells or neurons.

12. A method according to claim 8, in which the amyloid peptide is Aβ, and the method is performed in the presence of acetylcholinesterase (AChE).

13. A method according to claim 12, in which the AChE is added to the Aβ prior to its addition to the lipid preparation.

14. A method according to claim 13, in which the lipid preparation is coupled to a biosensor membrane.

15. A method according to claim 12, in which both AChE and Aβ are at a concentration of up to about 10 μM.

16. An agent for the treatment of a condition involving cerebral amyloidosis or cerebral angiopathy, which is a compound which has the ability to inhibit binding of an amyloid peptide to a lipid preparation which comprises cholesterol and phospholipid.

17. An agent according to claim 16, in which the amyloid peptide is aβ.

18. An agent according to claim 16, in which the compound also has the ability to inhibit cholesterol biosynthesis.

19. A composition for the treatment of a condition involving cerebral amyloidosis, cerebral angiopatby, or systemic amyloidosis, comprising a compound which has the ability to inhibit binding of an amyloid peptide to a lipid preparation which comprises cholesterol and phospholipid, together with a pharmaceutically acceptable carrier.

20. A method of treatment of a condition involving cerebral amyloidosis, cerebral angiopathy, or systemic amyloidosis, comprising the step of administering an effective amount of a compound which has the ability to inhibit binding of an amyloid peptide to a lipid preparation which comprises cholesterol and phospholipid to a subject in need of such treatment.

21. A method according to claim 20, in which the condition involves cerebral amyloidosis or cerebral angiopathy, and the amyloid peptide is Aβ.

22. A method according to claim 20, in which the condition involving cerebral amyloidosis or cerebral angiopathy is a sporadic condition such as Alzheimer's disease, amyloidosis associated with Down syndrome, prion-related cerebral amyloidosis, including Creutzfeld-Jacob disease and its new variant associated with “mad cow” disease, or sporadic cerebral angiopathy, or is a familial condition such as an autosomal dominant forms of familial Alzheimer's disease; hereditary cerebral haemorrhage associated with the Flemish, Arctic, Dutch, or Italian mutations of Aβ precursor protein; hereditary cerebral haemorrhage with amyloidosis (Icelandic type); meningocerebrovascular and oculoleptomeningeal amyloidosis; familial British dementia; familial Danish dementia; Cystatin C-related cerebral amyloid angiopathy; transthyretin-related cerebral amyloid angiopathy; and gelsolin-related spinal and cerebral amyloid angiopathy.

23. A method according to claim 20, in which the condition involving cerebral amyloidosis or cerebral angiopathy is sporadic or familial Alzheimer's disease, amyloidosis associated with Down syndrome, sporadic cerebral angiopathy, prion-related cerebral amyloidosis, familial British dementia, Cystatin C-related cerebral amyloid angiopathy, transthyretin-related cerebral amyloid angiopathy, or gelsolin-related spinal and cerebral amyloid angiopathy.

24. A method according to claim 20, in which the condition involves systemic amyloidosis, and is a primary amyloidosis, a reactive amyloidosis or a familial amyloidosis.

25. A method according to claim 21, in which
(a) the condition is immunoglobulin light chain-related (AL) amyloidosis, and the protein is immunoglobulin light chain or a biologically functional fragment thereof,
(b) the condition is amyloid protein A-associated amyloidosis and the amyloid protein is amyloid A; or
(c) the condition is familial amyloidosis, and the protein is selected from the group consisting of transthyretin, apolipoprotein A-I, gelsolin, fibrinogen A α, and lysozyme.

26. A method according to claim 20, in which the condition is associated with synuclein, and the condition is selected from the group consisting of Parkinson's disease, dementia with Lewy body formation, multiple system atrophy, Hallerboden-Spatz disease, and diffuse Lewy body disease.

27. A method according to claim 7 in which the lipid preparation is coupled to a biosensor membrane.

28. A method according to claim 27, in which the lipid preparation comprises about 30% to 80% cholesterol.

29. A method according to claim 27, in which the lipid preparation is a plasma membrane-enriched fraction of muscle cells or nerve cells.

30. A method according to claim 29, in which the cells are vascular smooth muscle cells or neurons.

31. A method according to claim 27, in which the amyloid peptide is Aβ, and the method is performed in the presence of acetylcholinesterase (AChE).

32. A method according to claim 31, in which the AChE is added to the Aβ prior to its addition to the lipid preparation.

33. A method according to claim 32, in which the lipid preparation is coupled to a biosensor membrane.

34. A method according to claim 31, in which both AChE and Aβ are at a concentration of up to about 10 μM.
